(19) United States
(12) Patent Application Publication
(10) Pub. No.: US 2014/0309188 A1
(43) Pub. Date: Oct. 16, 2014

(54) USE OF PHARMACEUTICAL COMPOSITION COMPRISING DEOXYNUCLEOSIDE AND NUCLEOSIDE FOR TREATMENT OF TUMOR

(71) Applicants: Shizhuang ZHANG, Weifang (CN); Xin CHENG, Weifang (CN); Zhiqin GAO, Weifang (CN); Ming HAN, Weifang (CN)

(72) Inventors: Shizhuang ZHANG, Weifang (CN); Xin CHENG, Weifang (CN); Zhiqin GAO, Weifang (CN); Ming HAN, Weifang (CN)

(21) Appl. No.: 14/254,911

(22) Filed: Apr. 17, 2014

Related U.S. Application Data
(63) Continuation-in-part of application No. PCT/CN2012/001392, filed on Oct. 16, 2012.

(30) Foreign Application Priority Data
Oct. 17, 2011 (CN) 201110314309.4

(51) Int. Cl. A61K 31/708; A61K 31/7068; A61K 31/7072; A61K 31/7076

(52) U.S. Cl. CPC A61K 31/708; A61K 31/7076; A61K 31/7068; A61K 31/7072
USPC 514/46; 514/45; 514/49; 514/50

(57) ABSTRACT

A method for treatment of a tumor including administering to a patient in need thereof a pharmaceutical preparation including a combination of a deoxynucleoside and a nucleoside. The deoxynucleoside is selected from deoxyadenosine, deoxyguanosine, deoxycytidine, thymidine, or a mixture thereof. The nucleoside is selected from adenosine, guanosine, cytidine, uridine, deoxyadenosine, deoxyguanosine, deoxycytidine, thymidine, or a mixture thereof.

### CROSS-REFERENCE TO RELATED APPLICATIONS

This application is a continuation-in-part of International Patent Application No. PCT/CN2012/001392 with an international filing date of Oct. 16, 2012, designating the United States, now pending, and further claims priority benefits to Chinese Patent Application No. 201110314309.4 filed Oct. 17, 2011. The contents of all of the aforementioned applications, including any intervening amendments thereto, are incorporated herein by reference.

### BACKGROUND OF THE INVENTION

1. Field of the Invention

The invention relates to a method for treatment of a tumor comprising administering to a patient in need thereof a pharmaceutical preparation comprising a combination of a deoxynucleoside and a nucleoside, and more particularly to the treatment of a tumor by using a combination of one or two deoxynucleosides and one or more nucleosides.

2. Description of the Related Art

Nucleoside includes ribonucleosides and deoxyribonucleosides. Including adenosine, guanosine, cytidine, and uridine, ribonucleosides are basic structural units for constituting RNA. Including deoxyadenosine, deoxyguanosine, deoxycytidine, and thymidine, deoxyribonucleosides are basic structural units for constituting DNA. Nucleoside can be obtained from the hydrolysis of nucleic acid. Ribonucleic acid (RNA) is hydrolyzed by an aqueous solution of pyridine, alumina, or enzyme to yield ribonucleosides. Deoxyribonucleic acid (DNA) is hydrolyzed by alumina or enzyme to yield deoxyribonucleosides. Nucleoside can also be obtained by chemical synthesis.

Deoxyribonucleoside monophosphates are basic structural units for forming DNA. Deoxynucleoside and its derivatives have good physiological activity and thus they are important raw materials for preparation of genetic medicine and for the study of genetic engineering researches. For example, 2-chlorodeoxyadenosine is used for the treatment of leukemia. 2′-deoxyadenosine and 3′-deoxyadenosine extracted from cordycepin have obvious inhibition against the growth of tumors. Deoxy acyclovir (Desciclovir) has significant antiviral activity. Dideoxycytidine (Zalcitabine) is widely used as a clinical drug for the treatment of AIDS. Thus, nucleoside analogues are good intermediates for preparation of antiviral drugs, antitumor drugs, and anti-AIDS drugs. However, there is no report in relation to the use of a combination of deoxyribonucleosides and/or other nucleosides for treatment of tumors.

### SUMMARY OF THE INVENTION

In view of the above-described problems, it is one objective of the invention to provide a method for treatment of a tumor comprising administering to a patient in need thereof a pharmaceutical preparation comprising a combination of a deoxynucleoside and a nucleoside. The combination comprises one or two of deoxynucleosides selected from deoxyadenosine, deoxyguanosine, deoxycytidine, and thymidine, and one or more of nucleosides selected from adenosine, guanosine, cytidine, uridine, deoxyadenosine, deoxyguanosine, deoxycytidine, and thymidine. The pharmaceutical preparation comprising the combination has excellent antitumor effect, reduced side effect, and delayed drug tolerance in contrast to the sole application of deoxynucleosides.

To achieve the above objective, in accordance with one embodiment of the invention, there is provided a method for treatment of a tumor comprising administering to a patient in need thereof a pharmaceutical preparation comprising a combination of a deoxynucleoside and a nucleoside. The deoxynucleoside is selected from deoxyadenosine, deoxyguanosine, deoxycytidine, thymidine, or a mixture thereof. The nucleoside is selected from adenosine, guanosine, cytidine, uridine, deoxyadenosine, deoxyguanosine, deoxycytidine, thymidine, or a mixture thereof.

In a class of this embodiment, the deoxynucleoside is selected from any one or two of the deoxyadenosine, deoxyguanosine, deoxycytidine, and thymidine, and the nucleoside is selected from any one of the adenosine, guanosine, cytidine, uridine, deoxyadenosine, deoxyguanosine, deoxycytidine, and thymidine, or a mixture thereof.

In a class of this embodiment, the deoxynucleoside is selected from any one of the deoxyadenosine, deoxyguanosine, deoxycytidine, and thymidine, and the nucleoside is selected from any one or two of the adenosine, guanosine, cytidine, uridine, deoxyadenosine, deoxyguanosine, deoxycytidine, and thymidine.

In a class of this embodiment, the deoxynucleoside is selected from any two of the deoxyadenosine, deoxyguanosine, deoxycytidine, and thymidine, and the nucleoside is selected from any one or two of the adenosine, guanosine, cytidine, uridine, deoxyadenosine, deoxyguanosine, deoxycytidine, and thymidine.

In a class of this embodiment, the deoxynucleoside is selected from any two of the deoxyadenosine, deoxyguanosine, deoxycytidine, and thymidine, and the nucleoside is selected from any one of the adenosine, guanosine, cytidine, uridine, deoxyadenosine, deoxyguanosine, deoxycytidine, and thymidine.

In a class of this embodiment, the deoxynucleoside is a mixture of the deoxyadenosine and the deoxyguanosine, and the nucleoside is a ribonucleoside.

In a class of this embodiment, the deoxynucleoside and the nucleoside have the same molar concentration.

In a class of this embodiment, the tumor is selected from the group consisting of gastric cancer, lung cancer, liver cancer, colon cancer, breast cancer, and reproductive system cancer.

Studies show that: compared with single nucleoside administration, the administration of the combination of different nucleosides reduces the single drug concentration in the blood, thereby weakening the side effect. In addition, the combination of different nucleosides can delay the occurrence of drug resistance of tumors. Compared with the administration of a combination of one deoxynucleoside and one nucleoside, the administration of a combination of one deoxynucleoside and two nucleoside significantly reduces the drug resistance. Compared with the administration of a combination of one deoxynucleoside and four nucleosides, the administration of a combination of one deoxynucleoside and two nucleoside, on one hand, significantly reduces the drug resistance, one the other hand, simplifies the preparation process and facilitates the application.

Preferably, the combination of the deoxynucleoside and the nucleoside is prepared as an injection for treatment of tumors. The injection is a conventional injection, or a slow release injection.

Different tumors have different nucleotide metabolism and composition characteristics, and thus different nucleosides have different inhibition strength against tumor cells. Studies show that, purine nucleoside has stronger anti-tumor activity than pyrimidine nucleoside, and inosine, which is a metabolite of purine nucleoside, also has some anti-tumor effects. Regarding the same base, the deoxynucleoside thereof has stronger anti-tumor activity than the nucleoside thereof. Deoxyadenosine, deoxyguanosine, deoxycytidine have one less oxygen atom in relation to corresponding nucleotides thereof, and the molecular weight of the former are 16D less than that of the latter. The deoxynucleosides have strong antitumor activity when the concentration thereof exceeds 0.03 mmol/L, and the tumor inhibition rate in vitro exceeds 80% when the concentration is 0.4 mmol/L. A single deoxynucleoside can relieve 15% of a solid tumor, with an effective percentage of exceeding 91.0%, and the usage dosage thereof is rather low, about between 4.0 and 35.0 mmol/kg/day, by intravenous injection.

Advantages according to embodiments of the invention are summarized as follows.

1. The combination of the deoxynucleoside and the nucleoside can be used for treatment of tumors comprising gastric cancer, lung cancer, liver cancer, colon cancer, breast cancer, and reproductive system cancer, with characteristics of high efficiency, broad spectrum, low toxicity, particularly no cytotoxicity to normal cells.

2. Compared with single nucleoside administration, the administration of the combination of different nucleosides reduces the single drug concentration in the blood, thereby weakening the side effect.

3. The combination of different nucleosides can delay the occurrence of drug resistance of tumors.

### DETAILED DESCRIPTION OF THE EMBODIMENT

For further illustrating the invention, experiments detailing a method for treatment of a tumor comprising administering to a patient in need thereof a pharmaceutical preparation comprising a combination of a deoxynucleoside and a nucleoside are described below. It should be noted that the following examples are intended to describe and not to limit the invention.

### Example 1

Growth Inhibition of Deoxynucleosides and Other Nucleosides on Human Tumor Cells

1. Experimental Method

Human hepatoma cells BEL-7402 and human lung cancer cells PG provided by Institute of Biochemistry and Cell Biology, SIBS, CAS were cultured in the culture medium RPMI 1640 comprising 10% fetal bovine serum. The cells in the exponential growth phase were collected, digested with 0.25% trypsin, inoculated to a 96-well plate with each well having 3×10³ cells, and then cultured in an incubator having 5% CO₂ at 37° C. for 24 hours. Each experimental group was provided with 8 wells. 60 μL of a nucleoside solution having a total concentration of 10.0 mmol/L was added to each well, and the culture medium was replenished to ensure each well to have a volume of 200 μL. The control group was administered with 5-FU having the same concentration as the nucleoside solution. The obtained cells were diluted with serum-free medium and filtered with sterile microporous membrane having an aperture size of 0.22 μm. 48 hours later, cell morphology was observed and photographed by an inverted microscope. Thereafter, 20 μL of MTT solution having a concentration of 5 mg/mL was added to each well of the 96-well plate, which was further cultured in the incubator having 5% CO₂ at 37° C. for 4 hours. After the culture, the supernate in the wells was removed. 150 μL of DMSO was added to each well. The 96-well plate was shaken for 10 minutes to facilitate the dissolution of the crystals. An automatic microplate reader (Thermo, Multiskan MK3) was employed to measure the absorbancy at 570 nm. Each experimental group was provided with 8 wells, and each experiment was repeated for three times. Table 1 lists the average value of the three experiments.

TABLE 1
Growth inhibition rate (%) of different nucleosides on
tumor cells Bel-7402 and PG after 48 hours' expermiments

| Group | A | G | C | U | dA | dG | dC | T | 5-FU |
|---|---|---|---|---|---|---|---|---|---|
| Hepatoma cells BEL-7402 | 86 | 83 | 65 | 49 | 96 | 95 | 86 | 87 | 91 |
| Lung cancer cells PG | 84 | 87 | 53 | 52 | 94 | 96 | 84 | 88 | 90 |

In Table 1, A, G, C, and U represent adenosine, guanosine, cytidine, and uridine, respectively, and dA, dG, dC, and T represent deoxyadenosine, deoxyguanosine, deoxycytidine, and thymidine, respectively.

The growth inhibition rate of the tumor cells is calculated as follows:

Survival rate of tumor cells (%)=measured OD value of experimental wells/OD value of wells of control group

Growth inhibition rate of tumor cells=100%−Survival rate of tumor cells (%)

2. Results and Analysis

As shown in Table 1, it can be concluded that:

1). Both deoxynucleosides and ribonucleosides have antitumor activity;

2). Purine nucleosides have stronger antitumor activity than pyrimidine nucleosides; and

3). Deoxynucleosides have stronger antitumor activity than ribonucleosides.

### Example 2

Growth Inhibition of a Combination of a Deoxynucleoside and a Nucleoside, and the Single Deoxynucleoside on Human Tumor Cells

1. Experimental Method

The experimental groups and the control group were administered with different nucleosides or a combination thereof, as listed in Table 2. Human colon cancer cell line HT-29 was provided by the Institute of Basic Medical Sciences (IBMS) of the Chinese Academy of Medical Sciences (CAMS). The experimental method was the same as that in Example 1. The concentrations of the nucleoside combination, the single deoxynucleoside, and 5-FU are all the same as that in Example 1.

TABLE 2
Growth inhibition rate (%) of different nucleosides
and combinations thereof on HT-29

| dA group | dA + G | dA + C | dA + U | dA + A | | dA + dC | dA | 5-FU |
|---|---|---|---|---|---|---|---|---|
| 48 hours | 95 | 86 | 87 | 93 | | 92 | 86 | 91 |
| dG group | dG + G | dG + C | dG + U | dG + A | dG + dA | dG + dC | dG | 5-FU |
| 48 hours | 93 | 87 | 85 | 96 | 95 | 91 | 91 | 93 |
| dC group | dC + G | dC + C | dC + U | dC + A | | | dC | 5-FU |
| 48 hours | 91 | 80 | 83 | 90 | | | 89 | 96 |
| T group | T + G | T + C | T + U | T + A | T + dA | T + dG | T | 5-FU |
| 48 hours | 92 | 82 | 80 | 91 | 87 | 86 | 85 | 95 |

2. Results and Analysis

As shown in Table 2, it can be concluded that: the combination of a deoxynucleoside and a nucleoside has stronger antitumor activity than a single deoxynucleoside. In addition, because the concentration of the single deoxynucleoside is decreased by 50%, the side effect of the pharmaceutical preparation is decreased accordingly.

### Example 3

Growth Inhibition of a Combination of one Deoxynucleoside and Two Nucleosides, the Single Deoxynucleoside, and a Combination of the Two Nucleosides on Human Tumor Cells

1. Experimental method: The experimental groups and the control group were administered with different nucleosides or a combination thereof, as listed in Table 3. Human hepatoma cells Bel-7402 was provided by Institute of Biochemistry and Cell Biology, SIBS, CAS. The experimental method was the same as that in Example 1. The concentrations of the nucleoside combination, the single nucleoside, and 5-FU are all the same as that in Example 1.

TABLE 3
Growth inhibition rate (%) of different deoxynucleosides
and combinations thereof on Bel-7402

| | dG + A + T | dG | A + T | 5-FU |
|---|---|---|---|---|
| 48 hours | 92 | 87 | 86 | 91 |
| 72 hours | 95 | 93 | 94 | 95 |

2. Results and Analysis

As shown in Table 3, it can be concluded that: the combination of one deoxynucleoside and two nucleosides has stronger antitumor activity against Bel-7402 than the single deoxynucleoside and the combination of the two nucleosides, and has low side effect.

### Example 4

Growth Inhibition of a Combination of One Deoxynucleoside and Two Nucleosides, the Single Deoxynucleoside, and a Combination of the Two Nucleosides on Human Gastric Cancer Cell Lines BGC823

1. Experimental Method

The experimental groups and the control group were administered with different nucleosides or a combination thereof, as listed in Table 4. Human gastric cancer cell lines BGC823 was provided by the Institute of Basic Medical Sciences (IBMS) of the Chinese Academy of Medical Sciences (CAMS). The experimental method was the same as that in Example 1. The concentrations of the nucleoside combination, the single deoxynucleoside, and 5-FU are all the same as that in Example 1.

TABLE 4
Growth inhibition rate (%) of different nucleosides
and combinations thereof on BGC823

| | dA + dG +C | dG + C | dA | dG + dC + U | dC + U | dG | dC + T + A | 5-FU |
|---|---|---|---|---|---|---|---|---|
| 48 hours | 95 | 90 | 89 | 90 | 83 | 85 | 88 | 91 |
| 72 hours | 98 | 93 | 91 | 94 | 89 | 90 | 91 | 95 |

2. Results and Analysis

As shown in Table 4, it can be concluded that:

1). The antitumor activities of a single deoxynucleoside, two nucleosides, and the combination of the single deoxynucleoside and the two nucleosides on human gastric cancer cell lines BGC823 are increasing successively. The combination of dA+dG+C comprising two kinds of purine deoxynucleosides has the strongest antitumor activity against BGC823.

2). The introduction of a nucleoside to a deoxynucleoside does not reduce the antitumor activity thereof, and on the contrary, the antitumor activity of the combination is improved.

### Example 5

Growth Inhibition of a Combination of Two Deoxynucleosides and One Nucleoside, a Combination of the Two Deoxynucleosides, and the Single Nucleoside on Human Breast Cancer Cell Lines Bcap-37

1. Experimental Method

The experimental groups and the control group were administered with different nucleosides or a combination thereof, as listed in Table 5. Human breast cancer cell lines Bcap-37 was provided by the Institute of Basic Medical Sciences (IBMS) of the Chinese Academy of Medical Sciences (CAMS). The experimental method was the same as that in Example 1. The concentrations of the nucleoside combination, the single nucleoside, and 5-FU are all the same as that in Example 1.

TABLE 5
Growth inhibition rate (%) of different nucleosides and combinations thereof on Bcap-37

| | dA + dG + C | dA + dG | C | dG + dC + U | dG + dC | U | dC + T + A | 5-FU |
|---|---|---|---|---|---|---|---|---|
| 48 hours | 93 | 90 | 65 | 89 | 80 | 58 | 85 | 92 |
| 72 hours | 96 | 93 | 71 | 92 | 85 | 67 | 90 | 95 |

2. Results and Analysis

As shown in Table 5, it can be concluded that:

1). The antitumor activities of a single nucleoside, two deoxynucleosides, and the combination of the two deoxynucleoside and the single nucleoside on human breast cancer cell lines Bcap-37 are increasing successively. The combination of the two deoxynucleosides and the single nucleoside has the strongest antitumor activity against Bcap-37.

2). The antitumor activity of the nucleoside combination is in relation to the characteristics of the tumors.

### Example 6

Growth Inhibition of a Combination of Two Deoxynucleoside and Two Nucleosides, a Combination of the Two Deoxynucleoside, and a Combination of the Two Nucleosides on Human Cervical Carcinoma Hela

1. Experimental Method

The experimental groups and the control group were administered with different nucleosides or a combination thereof, as listed in Table 6. Human cervical carcinoma Hela was provided by the Institute of Basic Medical Sciences (IBMS) of the Chinese Academy of Medical Sciences (CAMS). The experimental method was the same as that in Example 1. The concentrations of the nucleoside combination, the single nucleoside, and 5-FU are all the same as that in Example 1.

TABLE 6
Growth inhibition rate (%) of different nucleosides and combinations thereof on Hela

| | dA + dG + C + U | dA + dG | C + U | dG + dC + A + G | dG + dC | A + G | dC + T + G + C | 5-FU |
|---|---|---|---|---|---|---|---|---|
| 48 hours | 87 | 93 | 60 | 80 | 85 | 80 | 78 | 92 |
| 72 hours | 92 | 97 | 67 | 84 | 90 | 87 | 82 | 96 |

2. Results and Analysis

As shown in Table 6, it can be concluded that:

1). The combination comprising four nucleosides is not obviously superior to the combination comprising two nucleosides with regard to the antitumor activity.

2). The deoxynucleoside combination is superior to the nucleoside combination with regard to the antitumor activity against the human cervical carcinoma Hela.

### Example 7

Growth Inhibition of a Combination of Four Deoxynucleosides, and a Combination of Two Deoxynucleosides on Human Glioma Cell Line SHG-44

1. Experimental Method

The experimental groups and the control group were administered with different nucleosides or a combination thereof, as listed in Table 7. Human glioma cell line SHG-44 was provided by the Institute of Basic Medical Sciences (IBMS) of the Chinese Academy of Medical Sciences (CAMS). The experimental method was the same as that in Example 1. The concentrations of the nucleoside combination, the single deoxynucleoside, and 5-FU are all the same as that in Example 1.

TABLE 7
Growth inhibition rate (%) of different nucleosides
and combinations thereof on SHG-44

| | dA + dG + dC + T | dA + dG | dG + dC | dC + T | T + dA | 5-FU |
|---|---|---|---|---|---|---|
| 48 hours | 84 | 94 | 80 | 74 | 79 | 91 |
| 72 hours | 90 | 98 | 86 | 81 | 85 | 96 |

2. Results and Analysis

As shown in Table 7, it can be concluded that: 1). The combination comprising two deoxynucleosides is superior to the combination comprising four deoxynucleosides with regard to the antitumor activity. 2). The antitumor activity of the pyrimidine nucleoside is relatively low.

### Example 8

Growth Inhibition of a Combination of Two Deoxynucleosides and One Nucleoside at Different Dosages on Human Pancreatic Cancer Cell Line PANC-1

1. Experimental Method

The experimental groups and the control group were administered with a combination of two deoxynucleosides (dA+dG) and one nucleoside (C) with different dosages, as listed in Table 8. Human pancreatic cancer cell line PANC-1 was provided by the Institute of Basic Medical Sciences (IBMS) of the Chinese Academy of Medical Sciences (CAMS). The experimental method was the same as that in Example 1. The concentrations of the nucleoside combination and 5-FU are the same as that in Example 1.

TABLE 8
Growth inhibition rate (%) of a combination of two
deoxynucleosides and one nucleoside at different dosages
on human pancreatic cancer cell line PANC-1

| | A1 | A2 | A3 | A4 | A5 | A6 | A7 | 5-FU |
|---|---|---|---|---|---|---|---|---|
| 48 hours | 86 | 91 | 80 | 82 | 93 | 71 | 88 | 93 |
| 72 hours | 91 | 96 | 85 | 86 | 97 | 86 | 91 | 95 |

The dosage ratio of dA, dG, and C is 1:1:1 in the A1, is 2:2:1 in the A2, is 1:1:2 in the A3, is 2:1:2 in the A4, is 3:3:1 in the A5, is 1:1:3 in the A6, and is 3:1:3 in the A7.

2. Results and Analysis

As shown in Table 8, when the dosage ratio of dA, dG, and C of the combination comprising two deoxynucleosides and one nucleoside is 3:3:1, the antitumor activity is the best. In the preparation of clinical antitumor drugs, the feasibility and safety of clinical medication should be taken into account.

Through the observations on the antitumor effects of the deoxynucleosides and nucleosides, the combination of the deoxynucleosides and the nucleosides causes no side effect in contrast to a single deoxynucleoside or nucleoside.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for treatment of a tumor comprising administering to a patient in need thereof a pharmaceutical preparation comprising a combination of a deoxynucleoside and a nucleoside, wherein the deoxynucleoside is selected from deoxyadenosine, deoxyguanosine, deoxycytidine, thymidine, or a mixture thereof, and the nucleoside is selected from adenosine, guanosine, cytidine, uridine, deoxyadenosine, deoxyguanosine, deoxycytidine, thymidine, or a mixture thereof.

2. The method of claim 1, wherein the deoxynucleoside is selected from any one or two of the deoxyadenosine, deoxyguanosine, deoxycytidine, and thymidine, and the nucleoside is selected from any one of the adenosine, guanosine, cytidine, uridine, deoxyadenosine, deoxyguanosine, deoxycytidine, and thymidine, or a mixture thereof.

3. The method of claim 1, wherein the deoxynucleoside is selected from any one of the deoxyadenosine, deoxyguanosine, deoxycytidine, and thymidine, and the nucleoside is selected from any one or two of the adenosine, guanosine, cytidine, uridine, deoxyadenosine, deoxyguanosine, deoxycytidine, and thymidine.

4. The method of claim 1, wherein the deoxynucleoside is selected from any two of the deoxyadenosine, deoxyguanosine, deoxycytidine, and thymidine, and the nucleoside is selected from any one or two of the adenosine, guanosine, cytidine, uridine, deoxyadenosine, deoxyguanosine, deoxycytidine, and thymidine.

5. The method of claim 1, wherein the deoxynucleoside is selected from any two of the deoxyadenosine, deoxyguanosine, deoxycytidine, and thymidine, and the nucleoside is selected from any one of the adenosine, guanosine, cytidine, uridine, deoxyadenosine, deoxyguanosine, deoxycytidine, and thymidine.

6. The method of claim 5, wherein the deoxynucleoside is a mixture of the deoxyadenosine and the deoxyguanosine, and the nucleoside is a ribonucleoside.

7. The method of claims 1, wherein the deoxynucleoside and the nucleoside have the same molar concentration.

8. The method of claims 2, wherein the deoxynucleoside and the nucleoside have the same molar concentration.

9. The method of claims 3, wherein the deoxynucleoside and the nucleoside have the same molar concentration.

10. The method of claims 4, wherein the deoxynucleoside and the nucleoside have the same molar concentration.

11. The method of claims 5, wherein the deoxynucleoside and the nucleoside have the same molar concentration.

12. The method of claims 6, wherein the deoxynucleoside and the nucleoside have the same molar concentration.

13. The method of claim 1, wherein the tumor is selected from the group consisting of gastric cancer, lung cancer, liver cancer, colon cancer, breast cancer, and reproductive system cancer.

14. The method of claim 2, wherein the tumor is selected from the group consisting of gastric cancer, lung cancer, liver cancer, colon cancer, breast cancer, and reproductive system cancer.

15. The method of claim 3, wherein the tumor is selected from the group consisting of gastric cancer, lung cancer, liver cancer, colon cancer, breast cancer, and reproductive system cancer.

16. The method of claim 7, wherein the tumor is selected from the group consisting of gastric cancer, lung cancer, liver cancer, colon cancer, breast cancer, and reproductive system cancer.

17. The method of claim 8, wherein the tumor is selected from the group consisting of gastric cancer, lung cancer, liver cancer, colon cancer, breast cancer, and reproductive system cancer.

18. The method of claim 9, wherein the tumor is selected from the group consisting of gastric cancer, lung cancer, liver cancer, colon cancer, breast cancer, and reproductive system cancer.
